(19) United States
(12) Patent Application Publication
(10) Pub. No.: US 2024/0166575 A1
(43) Pub. Date: May 23, 2024

(54) METHOD FOR CONVERTING CARBON DIOXIDE INTO HIGH ADDED VALUE CHEMICAL COMPOUNDS THROUGH A MECHANOCHEMICAL PROCESS UNDER CONTINUOUS GAS FLOW CONDITIONS

(71) Applicant: UNIVERSITÀ DEGLI STUDI DI SASSARI, Sassari (SS) (IT)

(72) Inventors: Alessandro TARAS, Sassari (IT); Valeria FARINA, BOTTIDA (IT); Luca CAPPAI, Sassari (IT); Stefano ENZO, Sassari (IT); Sebastiano GARRONI, Sassari (IT); Gabriele MULAS, Sassari (IT)

(21) Appl. No.: 18/551,953

(22) PCT Filed: Mar. 17, 2022
(86) PCT No.: PCT/IB2022/052433

§ 371 Date: Sep. 22, 2023

(30) Foreign Application Priority Data
Mar. 24, 2021 (IT) 102021000007091

(51) Int. Cl. C07C 1/12; C01F 5/24; C01G 49/00

(52) U.S. Cl. CPC C07C 1/12; C01F 5/24; C01G 49/00; C07C 2521/06; C07C 2523/78

(57) ABSTRACT

The present invention relates to a method for converting carbon dioxide (CO₂) into high added value chemical compounds under continuous gas flow conditions. In particular, said process converts CO₂ into a mixture of high added value chemical compounds comprising low molecular weight hydrocarbons, mainly methane, ethylene and ethane, along with products of mineral carbonation, mainly Mg and Fe carbonates. Such CO₂ conversion is achieved through a mechanochemical process.

### TECHNICAL FIELD

The present invention relates to a method for converting carbon dioxide (CO₂) into high added value chemical compounds under continuous gas flow conditions. In particular, said method converts CO₂ into a mixture of high added value chemical compounds comprising low molecular weight hydrocarbons, mainly methane, ethylene and ethane, along with solid products of mineral carbonation, mainly Mg and Fe carbonates. Such CO₂ conversion is achieved through a mechanochemical process.

### BACKGROUND ART AND TECHNICAL PROBLEM

Carbon dioxide (CO₂) is a greenhouse gas which is released into the atmosphere as a consequence of natural phenomena (such as, for example, volcanic eruptions, cellular respiration, etc.) and human activities (such as, for example, processes of combustion of organic materials of fossil and non-fossil origin (wood, oil, petrol, diesel fuel, gas, biomass, etc.), deforestation activities, etc.), the concentration of which (>400 ppm since 2015) has exceeded by 45% the maximum historical values of the pre-industrial age.^{[1][2]}

Among the direct consequences of this increased CO₂ concentration, higher mean global temperature, the desertification of increasingly large areas, and the occurrence of “extreme” and potentially catastrophic climatic events are only some of the most important current environment-related issues on a global scale. Mitigating such effects requires timely interventions and positive actions by all industrialized countries.^{[3]} The carbon dioxide emission targets set by the Kyoto protocol in 1997, and then by the Paris agreement in 2015, in line with the targets of the later EU framework programmes for research and innovation (including Horizon 2020, Horizon Europe), have led the scientific community to studying new energy sources having a low environmental impact and developing new technologies and processes aimed at reducing the levels of carbon dioxide released into the atmosphere. Much attention is now being paid to reducing the use of fossil fuels, developing processes allowing for large-scale exploitation of renewable, or carbon-free, energy sources, and setting up processes and technologies for reducing the CO₂ concentration in the atmosphere. In this regard, the acronym CCUS (Carbon Capture, Utilization and Storage) refers in the literature to various technologies currently under development, but still far from possible large-scale application, which are devoted to reducing the atmospheric CO₂ concentration by capturing and storing CO₂ and transforming the captured carbon dioxide into products having a high commercial value.^{[4]}

In particular, much attention has been paid to Carbon Capture and Storage (CCS) procedures in recent years, with the goal of achieving, in the short term, a considerable reduction in carbon dioxide emissions by means of mineral carbonation processes occurring in geologically stable underground basins; however, the feasibility of such a large-scale process is currently limited by several factors, including, for example, the high costs to be incurred for sequestration, transportation and the energy necessary for injecting CO2-containing exhaust gas into the subsoil.^{[5]} On the other hand, the set of utilization processes (CCU) represents a virtuous way of controlling CO₂ concentration thanks to the possible creation of a so-called “zero carbon dioxide emission” scenario, since abundant CO₂ can be used as a raw material for the creation of a virtuous closed cycle, wherein the generated gas is transformed into valuable chemical compounds while at the same time balancing the atmospheric CO₂ levels.

In this latter respect, one aspect of exceptional interest from an application viewpoint concerns the experimental conditions necessary for activating the chemical processes for converting CO₂ into basic fuels and chemical products: literature data show that the activation and transformation of carbon dioxide (CO₂) require severe experimental conditions to overcome the slow reaction kinetics and the high energy barrier due to its thermodynamic stability (Δ_{f}G⁰ CO₂=−394.36 kJ mol⁻¹). In general, the conversion of carbon dioxide into basic fuels and chemical substances can be exemplified by means of redox processes occurring in the presence of molecular hydrogen. It should be taken into account that hydrogen, in its turn, has long been the subject of research for potential energy vectors alternative to fossil fuels, and the search for novel processes for producing it is still underway.^{[6]} The modes of CO₂ activation reported in the scientific literature refer to processes occurring in the presence of catalysts and promoted thermally or, to a limited extent, photochemically or electrochemically.^{[7]} Catalytic processes play a crucial role in the chemical industry and will be the key for all future scenarios aimed at implementing sustainable cycles; catalysts are, in fact, compounds capable of providing a reaction path that is alternative to the original reaction path and characterized by a significantly lower activation energy barrier.

The study of transformations of natural silicates with high Fe and Mg content like, for example, Olivine, which is a mineral widely available on the earth's crust^{[8]} that has a high potential for interaction with carbon dioxide^{[9]}, can be considered to be related to both of the above-mentioned lines of intervention (i.e. CCS and CCU). In particular pressure and temperature conditions, Olivine can, in fact, react with water (H₂O) to give the mineral known as serpentine (Mg₃Si₂O₅(OH)₄) plus a mixed oxide of Fe2+/Fe3+ and molecular H₂. This process, known as serpentinization, leads to molecular H₂and partial oxidation of Fe2+ and Fe3+ into magnetite oxide. In the presence of carbon dioxide, the molecular hydrogen thus formed can, through the Fischer-Tropsch process, form light hydrocarbons, including methane, or bring about CO₂ mineral carbonation processes. The reaction scheme can be summarized as follows:

α₁(Fe_{0.1}Mg_{0.9})SiO₄·nH₂O·nCO₂-->α₂Mg₃Si₂O₅(OH)+α₃Fe₃O₄+nH_{2↑}+α₄MgCO₃+nCH_{4↑}+minerals

In the above scheme, α₁-->α₄ are the stoichiometric coefficients of the phases that participate in the process. During this process, it is possible to observe both “precipitation” of CO₂ as solid carbonate and its transformation into methane and other low molecular weight hydrocarbons, made possible by the presence of molecular hydrogen (H₂) produced by the dissociation of H₂O occurring during the first step of the process. It should be noted that such processes already occur in nature, mainly in ocean floors, although with extremely slow reaction kinetics on a geological time scale, and have also been reproduced in the laboratory, although in the presence of particularly high pressure and temperature conditions.^{[10]}

Considering the conditions required for the activation of the above process by thermal means, the use of less conventional forms of energy, e.g. kinetic energy, may offer some specific solutions of interest. In particular, the study of the transformations induced in chemical systems comprising at least one solid phase by supplying mechanical energy is generally referred to as “mechanochemistry”: such transformations are achieved through the use of particular apparatuses, i.e. the so-called mechanochemical reactors, which may have different construction specifications and perform different mechanical actions. For example, high-energy micromills or rotary mills provided with milling means (such as balls made of suitable materials and having variable sizes) are examples of apparatuses that are commonly employed, on a laboratory scale, for the execution of mechanochemical processes. However, types of devices provided with milling means can be used as well for executing mechanochemical processes. The continuous repetition of processes of breaking and comminution of the solid phases involved, the renewal of the exposed surfaces of the mechanically processed phases, and the local conditions achieved often make it possible to successfully obtain chemical transformations that would not be otherwise attainable by thermal treatment. The versatility of these mechanical processes and the specific conditions that can be obtained in the interaction between solid phases and gaseous phases, as well as the wide industrial diffusion of processes involving solid phases, support the study of this mode of activation of mechanochemical processes for CO₂ conversion, for which, however, scientific literature data are still very scant and lack a global vision that may clarify their feasibility, specificity and possible application advantages.

As already highlighted above, aiming at contributing to mitigating climate changes by reducing CO₂ emissions, the Carbon Capture Storage (CCS) and the more recent Carbon Capture Utilization (CCU) strategies have drawn the attention of scientists all over the world. Mineral carbonation, also known as “mineral sequestration”, is the most renowned and widespread CO₂ storage technique among such processes. The main advantage of mineral carbonation is the formation of stable carbonates capable of “storing” CO₂ for long periods, thanks to its conversion into carbonate by reaction with metal oxides. As aforementioned, Olivine and Serpentine are the most promising minerals for carbon sequestration, because of their high magnesium content. In this frame, the CO₂ absorption process occurring during olivine serpentinization has been studied experimentally, but literature data are not homogeneous and mostly refer to the effects of mechanical activation upon olivine, focusing mainly on its structural and superficial transformation, for the purpose of promoting CO₂ storage [R. A. Kleiv et al. (2006), Mechanical activation of olivine. Miner. Eng. 19, 340-347; P. Baláz̆ et al. (2008), Structural changes in olivine (Mg, Fe)2SiO4 mechanically activated in high-energy mills. Int. J. Miner. Process. 88, 1-6; M. Fabian et al. (2010), The influence of attrition milling on carbon dioxide sequestration on magnesium-iron silicate. Miner. Eng. 23, 616-620; K. L. Sandvik et al. (2011), Mechanically activated minerals as a sink for CO₂. Adv. Powder Technol. 22, 416-421; I. M. Power et al. (2013), Serpentinite carbonation for CO₂ sequestration. Elements 9, 115-121; E. Turianicová et al. (2013), A comparison of the reactivity of activated and non-activated olivine with CO₂. Int. J. Miner. Process. 123, 73-77; J. Li et al. (2015), Ultra-fine grinding and mechanical activation of mine waste rock using a high-speed stirred mill for mineral carbonation. Int. J. Miner. Metall. Mater. 22, 1005-1016; I. Rigopoulos et al. (2015), Enhancing the rate of ex situ mineral carbonation in dunites via ball milling. Adv. Powder Technol. 27, 360-371]. In particular, it has been demonstrated that mechanical grinding of Olivine significantly increases its ability to form iron and magnesium carbonates, making CO₂ capture a potential method of stable long-term storage. Mechanical activation has proved to be even more effective when the treatment occurs in the presence of liquids like water [E. Turianicová et al. (2008), A possible way to storage carbon dioxide on mechanically activated olivine (Mg, Fe)2SiO4. VI Int. Conf. Mechanochemistry Mech. Alloy. 316-319] or ethanol [I. Rigopoulos et al. (2015), Carbon dioxide storage in olivine basalts: Effect of ball milling process. Powder Technol. 273, 220-229], as a consequence of the larger surface generated in humid conditions. Such studies have not paid much attention to the chemical reduction of CO₂ for producing methane and light hydrocarbons.

In general, in fact, the currently available technologies that concern the transformation of CO₂ into methane are mostly based on thermally activated processes or on processes that exploit the activity of special bacteria. No examples exist in the literature which refer to the transformation of CO₂ into hydrocarbons, such as, for example, methane and other light hydrocarbons like ethane or ethylene, through the use of mechanochemical activation methods. Also, there are no documents relating to the transformation of carbon dioxide into methane and light hydrocarbons through the use of natural minerals like Olivine, in a process exploiting a mechanical method to achieve CO₂ transformation.

### Drawbacks of the Prior Art

Notwithstanding the importance of the above-illustrated themes from an environmental and economical viewpoint, to the present inventors' knowledge no consolidated and commercially available technologies currently exist which are applicable at an industrial level for controlling the processes of capturing, storing and converting atmospheric CO₂: the extended research activity of the scientific community in this field, referable to the above-mentioned CCS (Carbon Capture and Storage) and CCU (Carbon Capture and Utilization) processes, has been mostly directed to studying, respectively, mineral carbonation processes and confinement on geological sites and conversion processes via high-temperature catalytic treatments. In this latter case, the focus is mainly on setting up complex catalytic systems, often obtained starting from precious metals and other chemical systems, via procedures referred to as fine chemistry, and such catalysts generally cannot be easily and economically produced and used on an industrial scale. Furthermore, the operating conditions required by the conversion processes, also in the presence of the above-mentioned catalytic systems, are far from standard conditions: the temperatures of the processes for transforming CO₂ into fuels, studied in pre-application conditions, are generally >300° C. and represent themselves a source of high energy consumption and environmental pollution.

Lastly, it must be underlined that no technologies are currently available which are based on the transfer of mechanical energy for activating the above-mentioned capture, storage and utilization processes.

Another aspect must also be pointed out which has not been taken into account in previous studies, while however being connected with the experimental/actual conditions under which CO₂ is released into the atmosphere. When not related to natural phenomena, the processes of CO₂ production are connected to processes of combustion of organic materials used on an industrial scale, generally fossil fuels (but this also applies to combustion of biomass, wood, coal, etc.) such as, for example, energy production processes in thermoelectric power plants, iron and cement production, processes related to automotive systems, as well as air-conditioning processes, feeding processes, etc. The experimental conditions under which the combustion phenomena occur are far from standard (e.g. the combustion of fossil materials in the boilers of thermoelectric power plants reaches temperature values of approximately 560° C., i.e. much higher than ambient temperature, and pressure values much higher than atmospheric pressure), but the produced flow of CO₂ is generally released into the atmosphere at a pressure only slightly higher than atmospheric pressure and at a temperature of approximately ≤100° C. Merely by way of example, the fumes exiting the post-combustion chimney in the typical conditions of a thermoelectric power plant are characterized by temperature values of approximately ≤100° C. (90-95° C.) and a pressure close to atmospheric pressure (approx. 100,000 Pa), and their composition generally includes N₂ (approx. 75%), O₂ (approx. 9%), H₂O (approx. 6%), CO₂ (approx. 9%). Therefore, the fumes consist of a humid mixture containing excess thermal energy and kinetic energy, and are released into the atmosphere under continuous flow conditions, at a velocity of 10 to 20 m/s, and with flow rate values that depend on the size of the chimney. Conditions not too different from these can be found in other industrial chimneys and in downstream exhaust systems of other processes of combustion of fossil materials.

### Technical Problem

In light of the above discussion, it has become particularly important to optimize processes of CO₂ conversion induced by mechanical activation under continuous gas flow conditions, in particular as a gas mixture having a CO₂ content (approx. 8-10%) comparable to the above-mentioned data.

Therefore, the need is still felt by those skilled in the art for an economical and environmentally compatible method of converting carbon dioxide (CO₂), either as such or mixed with other gases, into high added value chemical compounds under continuous gas flow conditions.

The object of the present invention is to provide an adequate response to the above-highlighted technical problem.

### Description of the Invention

To such end, the present inventors' attention was focused on processes for the production of hydrogen from water and for CO₂ conversion induced by mechanical activation in the presence of mineral Olivine and H₂O, and particular attention was paid by the same inventors to studying/optimizing the experimental conditions of the process, so as to make them compatible with the implementation conditions of several industrial-scale processes producing large amounts of CO₂. In this regard, a number of preliminary laboratory tests were also carried out, which showed the great potential of olivine in the evolutionary process of molecular hydrogen obtained by mechanochemically activated dissociation of water, as well as in the production of fair amounts of methane. Said preliminary tests were conducted using a mechanochemical reactor in “batch” (discontinuous) mode, and made it possible to evaluate, among other things, some aspects related to the structural evolution of the solid phases involved and to the kinetics of the process under specific experimental conditions.^{[11]}

The present invention relates, on the other hand, to a chemical process, conducted by mechanical activation under continuous gas flow conditions, for converting CO₂ into a mixture of high added value chemical compounds comprising a mixture of low molecular weight hydrocarbons, mainly methane, ethylene and ethane, along with solid products of mineral carbonation, mainly Mg and Fe carbonates. Therefore, CO₂ conversion is effected by means of a continuous mechanochemical process under gas flow, in the absence of any solvents, starting from the following reagents: Olivine powder (solid solution of Mg and Fe silicates), H₂O (whether or not deionized) and CO₂ (or a gas flow containing CO₂ in quantities (approx. 8-10%) similar to those previously specified herein for fumes released from post-combustion chimneys of an industrial manufacturing process or the like). The solid reagents are inserted into a suitable mechanochemical reactor constructed (by way of non-limiting example, as previously described) starting from a ball mill driven by, for example, an electric motor (other driving means may however be used, as will be specified hereinafter). The solid phases are subjected to mechanical treatment by suitable milling bodies (e.g. balls made of steel or other suitable materials, or other milling means adequate for the type of reactor in use), and are put under conditions of reaction with the fluidic reagents (CO₂, H₂O, or a flow of humid gases containing CO₂), which in turn are transported into and through the reactor under continuous flow conditions.

The process is characterized by the absence of any solvents, by the method of activation of the process (using mechanical energy supplied by the milling bodies to the reagents that are trapped during ball-ball or ball-jar collisions), by very high yield values of the CO₂ transformation process, and by very fast reaction kinetics, in addition to rather mild process conditions (e.g. temperature and pressure) comparable to those described above with reference to fumes released from post-combustion chimneys of an industrial manufacturing process or a process of combustion of organic materials (e.g. wood, coal, oil, petrol, diesel fuel, gas and/or biomass).

Such characteristics lend environmental sustainability properties to the process of the invention, resulting in great potential interest in the commercial exploitation of said process.

The mechanism through which the series of chemical transformations of the process occur is certainly complex and comprises several stages, including dissociation of the H₂O molecule, dissociation of the CO₂ molecule, surface adsorption processes, chemical absorption processes, bulk scattering, structural evolution of the solid phases, synthesis of hydrocarbons, processes of desorption of gaseous phases from the surfaces of the solids.

A detailed evaluation of such aspects goes beyond the application properties related to the scope of the present invention; it is however of interest here to underline that during the initial phases of the process large amounts of H₂are produced, i.e. hydrogen in molecular form (with reference to the reaction under examination), originated from the dissociation of water promoted by the kinetic energy derived from the impact of the milling bodies against the humid Olivine powder. Formation of H₂is also correlated with oxidation processes (Fe2+ to give Fe3+) that can be observed in the solid phases that constitute Olivine.

Once hydrogen has been produced, in the presence of CO₂ it shows to be able to induce carbon dioxide reduction, again thanks to the mechanical energy generated and transferred during the non-elastic collisions occurring between the milling bodies, the olivine powder and the reagents in liquid and gaseous phase.

In summary, it is the object of the present invention to provide a continuous mechanochemical process under gas flow to carry out the conversion of CO₂ into a mixture of high added value chemical compounds, said mixture comprising a mixture of low molecular weight hydrocarbons, mainly methane, ethylene and ethane, and solid products of mineral carbonation, mainly Mg and Fe carbonates, said process comprising at least the following steps:
a)—continuously passing a gas flow comprising, or consisting of, CO₂ through a reactor equipped with milling means, and in the presence of Olivine powder and water, in liquid or gaseous form, at from room temperature to ≤100° C.; said reactor being subjected to motion in such a way that the milling means trigger a mechanochemical reaction that, on one side, produces hydrogen from the present water, and then said hydrogen carries out the conversion of a portion of CO₂ into said low molecular weight hydrocarbons, while, on the other side, the other portion of CO₂ reacts with the transformation products of Olivine and non-reacted H₂O to give said products of mineral carbonation;
b)—separating and recovering from the gas mixture exiting the reactor said low molecular weight hydrocarbons and non-reacted hydrogen obtained in step a);
c)—at the end of the reaction, separating and recovering from the reactor said solid products of mineral carbonation of olivine obtained in step a).

The advantages of the technique (process) of the present invention, as described above, include at least:
A) use of a low-cost natural mineral (Olivine: about 13 USD/ton);
B) generation of the hydrogen necessary for the process of chemical reduction of carbon dioxide into methane according to the following reaction scheme:

CO₂+4H₂-->CH₄+2H₂O

directly from the water contained in the gas mixture;
C) execution of the conversion process at a low temperature (from room temperature to approx. ≤100° C.; preferably, to approx. ≤80° C.; more preferably, to approx. ≤60° C. or ≤40° C.);
D) further use of the solid materials thus obtained, which, once exhausted, are inert in terms of toxicity and chemical noxiousness, and any metals that may be present therein can be extracted using methods known in the literature; they are also totally reusable in the cement industry (e.g. to give magnesia cement), thus not representing a problem for the environment;
E) production of energy vectors (hydrogen and methane and light hydrocarbons, e.g. ethane and ethylene) and construction materials (magnesium oxide-magnesia cement).

### BRIEF DESCRIPTION OF THE FIGURES

FIG. 1 shows an illustrative embodiment of the experimental apparatus of the present invention.

FIG. 2 represents the relationship between conversion (X), selectivity (S) and yield (Y) when products B and C are obtained from the reagents, indicated as A, plus a certain quantity of non-reacted reagent A. At time t1 the process has not yet begun, and only the reagents (A) are present. At time t2, following the start of the process, the system will be composed of the products (B and C) and a part of reagents (A) not yet reacted.

FIGS. 3a) and 3b) show, respectively, the CO₂ conversion rate and the concentration, expressed in ppm, of the products obtained, as recorded during the grinding process.

FIGS. 4a) and 4b) show, respectively, the selectivity of the products obtained during the grinding process and the estimated yield of the products.

FIG. 5 shows a comparison among tests conducted with, respectively, 0.15, 0.3 and 0.6 ml of H₂O and 2 g of Olivine.

FIG. 6 shows the CO₂ conversion rate, evaluated by using milling bodies having different masses.

FIG. 7 shows CO₂ conversion as a function of grinding time. The two curves refer to tests conducted with different values of the revolution speed of the mill, as indicated in the box.

FIG. 8 represents a comparison that shows CO₂ conversion as a function of time with Olivine at its first use (grey curve) and with Olivine at its second use (black curve).

FIG. 9 shows the diffraction patterns of unprocessed Olivine (bottom) and of the post-reaction materials treated under different conditions, as described in this specification.

FIG. 10 shows the diffraction patterns of unprocessed Olivine (bottom) and of a representative sample of processed Olivine (top). At its base, the graph shows the sequences of the characteristic signals of the main crystallographic phases involved.

FIGS. 11A and 11B show, respectively, an SEM micrography of a sample of Olivine pre-ground for 1 hour and an SEM micrography of a sample of Olivine processed in the presence of water and carbon dioxide for a time longer than one hour.

FIG. 12 shows the map of elements and the EDX microanalysis. The upper part shows the map of elements generated a sample of on Olivine processed by mechanochemical activation with water and carbon dioxide. The lower part shows the fluorescence spectrum, with the characteristic signals of the existing elements. The box shows the mean percentages of the detected elements.

FIG. 13 shows the FT-IR spectra of unprocessed and processed (i.e. subjected to mechanochemical 1 reaction) Olivine. Prior to recording the spectra, the samples were dried in a stove at 80° C. to remove all traces of humidity.

FIG. 14 shows the evolution of the non-reacted hydrogen produced during the CO₂ activation process.

### EXPERIMENTAL SECTION

### Description of the Reactor and Reaction Conditions

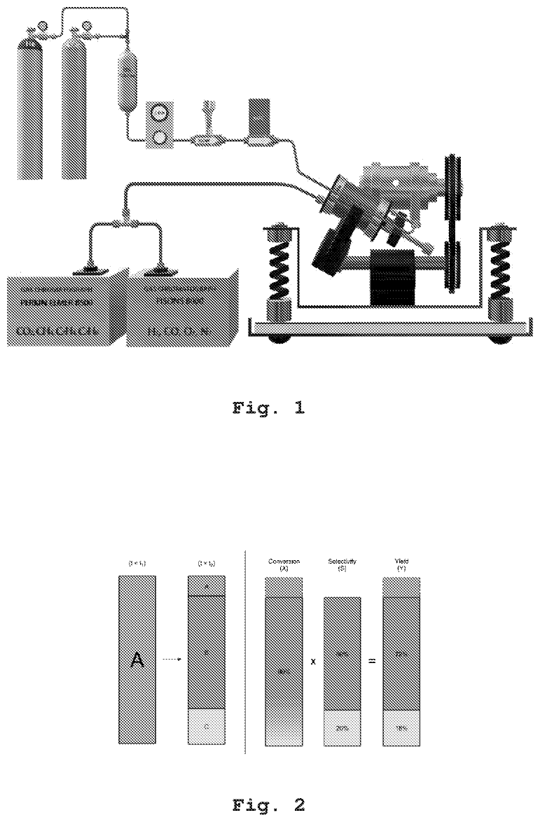

The following experimental section will describe, by way of illustrative example, some of the characteristic aspects of the present invention, without however by no means limiting the broad application potential thereof. Based on the following explanations, those skilled in the art will have no difficulty in applying and modifying the teachings provided herein by adapting the dimensions, quantities and construction aspects to specific practical implementations. In one embodiment, the equipment used for the experimental activity is schematized in FIG. 1. It essentially consists of a cylindrical reactor (a grinding jar (mill) having an internal volume of approx. 65 ml), suitable for the execution of mechanical treatments by means of ball milling processes. The reactor and the milling bodies are made of hardened stainless steel (of course, the dimensions and materials of the reactor may be freely selected and/or defined for specific applications, depending on the type and rate of gas flow (e.g. an exhaust gas) to be treated. The same also applies to the number, size and material of the milling means). The cylindrical reactor is equipped with gas-tight covers and gas transfer lines. The gas lines, e.g. partly made of steel and partly consisting of flexible Teflon (PTFE) hoses, provide the connection between the reagent gas supply (or the exhaust gas flow) and the reactor, and between the latter and the two gas chromatographs for analyzing the reagent and the produced gases. Such a “flow” configuration of the reactor allows the execution of tests under a continuous supply of gaseous reagents. The gas chromatographs are both equipped with detectors suitable for a quantitative evaluation of the gaseous phases of interest. A first gas chromatograph, equipped with a GS-Q wide-bore capillary column and TCD-FID detectors, with He as a carrier gas, made it possible to evaluate the CO₂ through the TCD detector and the low molecular weight hydrocarbons through the FID detector; a second gas chromatograph, equipped with a column packed with 13× (10 Å) molecular sieves and a TCD detector, with Ar as a carrier gas, made it possible to evaluate the amounts of H₂, CO, O₂ and N₂.

The mechanochemical reactor was housed in the seat of a known Spex Mixer/Mill mod.8000, [Spex Certiprep., https://www.spexcertiprep.com], suitably modified and equipped with a three-phase electric motor controlled by a speed regulator, which provides control over the number of revolutions of the motor shaft and imparts to the mill a three-dimensional motion in space. The tests were conducted under two different conditions: at 875 and 1,000 revolutions per minute (rpm) of the motor shaft, respectively corresponding to 14.5 and 16.6 Hz.

In practice, the revolution speed of the reactor can be varied, depending on the type of application, from 500 rpm to 1,500 rpm; preferably, from 600 rpm to 1,400 rpm; more preferably, from 700 rpm to 1,300 rpm; even more preferably, from 800 rpm to 1,200 rpm.

The tests were conducted using solid samples having a mass of 2 g of commercial Olivine (produced by SATEF-HA, Italy). The latter was subjected to a mechanical pre-treatment in Air atmosphere for 1 h using 3 balls (made of stainless steel) having a mass of 4 g each, for the purpose of homogenizing the powder size. At the end of the pre-treatment, 0.3 ml of deionized H₂O were added to the Olivine powder.

The above-described conditions correspond to an Olivine/H₂O stoichiometric ratio of 1:2.

The reactor was connected to the gas lines and housed in the support of the Spex 8000 commercial mill, for the execution of the reactive tests.

The gaseous reaction mixture was composed of CO₂ (Sapio, purity of 99.995), with abundance of 10% by volume in He (Sapio, BIP purity). After saturating the atmosphere of the mechanochemical reactor, the reactive mixture was fed in a flow of 10 ml/min, kept constant through a regulation valve and measured with a mass flowmeter (in the applications of the process of the invention, the flow rate may be changed freely according to the amount of exhaust fumes containing CO₂ released per time unit, the chimney size, and other parameters related to the release of said fumes). The composition of the reactive mixture for the tests was selected in tight relation with the average composition data of the mixture containing CO₂ exiting the above-mentioned industrial combustion processes. In these tests, the matrix was He, instead of N₂, in order to avoid any side effects due to a possible interaction between N₂ and Olivine and to avoid any overlapping effects in the analyses of the permanent gases. This will not limit the applicability of the invention, which may also be implemented by the operator with N₂ as a gaseous matrix.

### Description of the Results of the CO₂ Conversion Tests and of the Analyses of the Solid and Gaseous Phases

The following will present the results of the CO₂ conversion tests under the above-described conditions. Data will be provided about the analyses of the gaseous phases conducted by gas chromatography, the analyses of the solid phases conducted by X-ray diffraction techniques, and the morphological and elementary analyses conducted on the solid phases by, respectively, scanning electron microscopy (SEM) and X fluorescence.

The mechanical treatment in reactive atmosphere was conducted by using 3 steel balls having a mass of 4 g each and by setting the revolution speed of the motor to 875 rpm, unless otherwise specified. The kinetics of the CO₂ conversion process was monitored by evaluating the degree of CO₂ conversion via measurement of the concentration of CO₂ eluted by the reactor after selected mechanical treatment times up to 180 minutes.

### Definition of the Descriptor Parameters of the Process

The study of the process was conducted by monitoring, as parameters, the rate of CO₂ “conversion”, the “selectivity” in giving the products, and the “yield” of a specific product. FIG. 2 illustrates, in graphic form, the meaning of these three descriptor parameters.

### CO2 Percent Conversion

It indicates the rate of CO₂ transformation compared to the initial concentration, and is expressed by the following mathematical relation:

$$C {O}_{2} conversion , \% = \frac{{[ C {O}_{2} ]}_{i} - {[ C {O}_{2} ]}_{t}}{{[ C {O}_{2} ]}_{i}} \cdot 100$$

where “[CO₂]ᵢ” indicates the CO₂ concentration at time “i=initial”, i.e. before the start of the process;
where “[CO₂]ₜ” indicates the CO₂ concentration at time “t”, i.e. at a time instant after the start of the process.

In gas chromatography, the concentration of an analyte can be expressed as the product of a response factor and the area of the signal of the analyte under evaluation. From a mathematical viewpoint, this can be expressed as:

[CO₂]=f_{f}·Area of CO₂ signal
where “[CO₂]” is the concentration of the carbon dioxide analyte; and
where “fᵣ” is the response factor for the CO₂ analyte and refers to a specific column and a specific method of analysis.

Therefore, with appropriate substitutions and simplifications, the equation for calculating the CO₂ conversion rate can be rewritten as:

$$CO 2 conversion , \% = \frac{( {f}_{r} \cdot Area {CO}_{{2}_{i}} ) - ( {f}_{r} \cdot Area {CO}_{{2}_{t}} )}{( {f}_{r} \cdot Area {CO}_{{2}_{i}} )} \cdot 100$$

$$CO 2 conversion , \% = \frac{{f}_{r} \cdot ( Area {CO}_{{2}_{i}} - Area {CO}_{{2}_{t}} )}{{f}_{r} \cdot Area {CO}_{{2}_{i}}} \cdot 100$$

$$C {O}_{2} conversion , \% = \frac{Area {CO}_{{2}_{i}} - Area {CO}_{{2}_{t}}}{Area {CO}_{{2}_{i}}} \cdot 100$$

### Selectivity

It expresses the ratio between the concentration of a specific product and the total product concentration. It can be calculated by means of the following mathematical relation (expressed for methane):

$$C {H}_{4} Selectivity \% = \frac{[ C {H}_{4} ]}{⌈ X ⌉} \cdot 100 ;$$

$$[ X ] = total product concentration$$

This relation is applied in order to obtain the selectivity of the detectable products, taking into account that, for each one of them, it will be necessary to draw a calibration straight line in order to be able to obtain the concentration.

### Yield of the Reaction in Giving Methane (and/or Other Products)

It expresses the percent abundance of a product relative to the conversion rate of the process. From a mathematical viewpoint, it can be expressed as the product of selectivity and conversion:

CH₄ Yield, %=CH₄ Selectivity−CO₂ Conversion/100

### Results

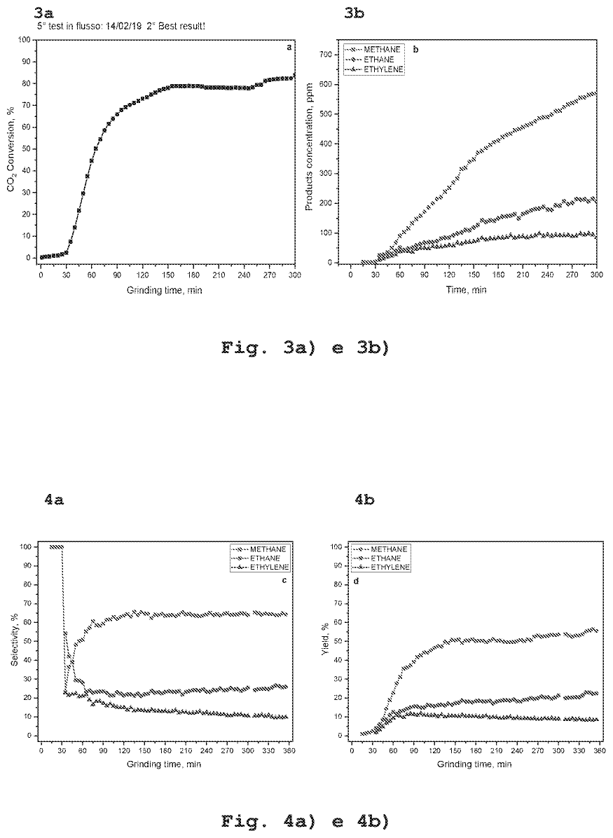

The test results, expressed as percent values of CO₂ conversion over time (FIG. 3a), highlighted very good reproducibility characteristics, with a variability percentage of less than 5% (attributable to the experimental/instrumental error), and FIG. 3a) shows a typical profile of the experimental data of the reaction kinetics. The pattern shows a sigmoidal CO₂ conversion profile. In all tests carried out, conversion grows significantly only after an initial induction time, which in the test shown in FIG. 3a) was approximately 30 minutes. Conversion speed then increases, reaches a maximum value at an inflection point in the curve, and then decreases, corresponding to the asymptote the conversion tends to, which in the case represented in FIG. 3a) exceeds the value of 80%. As can be seen in FIG. 3a), 150 minutes after the start of the grinding process the CO₂ conversion rate remained almost constant for 3 hours. The sigmoidal pattern of the kinetic curve is typical of many mechanochemical processes, and particularly of processes involving solid-gas heterogeneous systems. Such a pattern has often been interpreted as the result of nucleation-and-growth processes and analyzed on the basis of Avrami-Erofeev mechanisms. Without going into details of the mechanistic analysis, it is however useful to underline, in this context, that the shape of the kinetic curve profile comprises multiple elementary stages of the conversion process, including superficial H₂O adsorption by Olivine, its dissociation, processes of H₂ and O₂ scattering in the bulk of the solid phase, oxidative processes undergone by Fe2+, adsorption and absorption of CO₂, its reduction by H₂, mineral carbonation processes, scattering and desorption of the formed hydrocarbons, etc. The exact correlation between such processes and the shape of the curve goes beyond the intention of the present invention, but they should be taken into consideration for a global evaluation of the process. A further aspect is the analysis of the hydrocarbon phases produced: the process highlights how a CO₂ fraction undergoes a reduction process and hence can be transformed into light hydrocarbons such as methane, ethylene and ethane. The kinetic curves concerning the formation of such gaseous phases are shown in FIG. 3b): the profile of such curves seems to follow the same sigmoidal pattern as the one previously mentioned, and the concentration values of said phases different. The maximum concentrations measured for the three above-mentioned products are 680 ppm for methane, 280 ppm for ethane and 100 ppm for ethylene, measured after 330 minutes of grinding time (FIG. 3b)). FIG. 4a) shows the trend over time of the selectivity data for the above-mentioned products: methane is the main product, with a selectivity of 60%, while the selectivity values for ethane and ethylene are, respectively, 20-25% and 8-10%.

FIG. 4b shows global yield data, indicating a production of approximately 50% for methane, 10-15% for ethane and 10% for ethylene.

It must be pointed that the conversion, selectivity and yield data are very stable over time, with values reaching a maximum point after approximately 90 minutes and then remaining constant (except for the experimental error) throughout the observation interval, i.e. for up to 360 minutes of mechanical treatment.

In order to deepen the knowledge of the process mechanism and determine the experimental conditions that led to the best results in terms of conversion, selectivity and yield, the process was studied as a function of some experimental parameters, in particular the stoichiometric ratio between H₂O and Olivine and the mechanical energy transferred to the reagents during the mechanical treatment.

### Dependency of the Kinetics on the Olivine/H₂O Stoichiometric Ratio

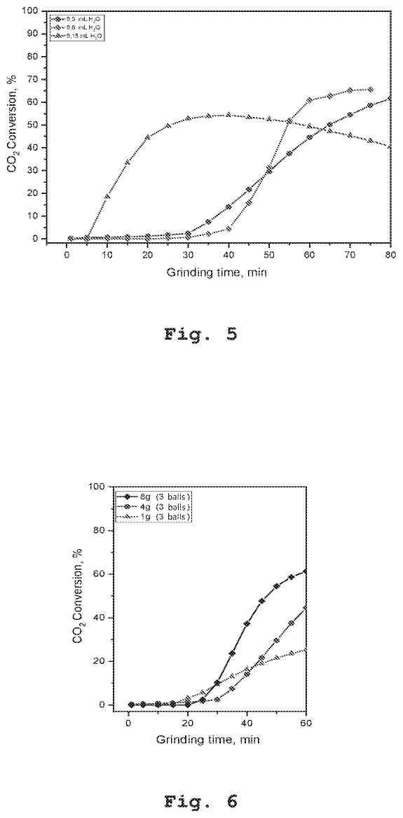

The H₂O content in the reaction environment represents an important parameter in the reaction of methanation of CO₂ mechanically activated in the presence of Olivine, because water is the hydrogen source. FIG. 5 shows the results of tests conducted with different H₂O contents in the reaction environment.

The corresponding stoichiometric ratio values are as follows:
0.15 ml H₂O, Olivine/H₂O=0.01 mol Olivine/0.01 mol H₂O=1:1* *Such stoichiometric ratios were calculated considering Olivine powders as consisting only of Forsterite Ferroan phase of formula (Fe_{0.184}, Mg_{1.816})SiO₄ and molar mass of 146.4693 g/mol.
0.3 ml H₂O, Olivine/H₂O=0.01 mol Olivine/0.02 mol H₂O=1:2*
0.6 ml H₂O, Olivine/H₂O=0.01 mol Olivine/0.03 mol H₂O=1:3*

The kinetic curves shown in FIG. 5 highlight the existence of an initial period during which CO₂ conversion appears to be negligible. Such induction period seems to be influenced by the adopted experimental conditions, and its amplitude depends on the relative ratio between Olivine and H₂O. In the three tests presented herein, therefore, the conversion data increase according to a sigmoidal kinetic profile, whose specific characteristics seem to differ (maximum conversion speed values and mechanical treatment time values at which measurements were taken). The maximum conversion value obtained during the test appears to be similar (approx. 60%) in all three cases. It should be noted that, in the test conducted with the lowest (1:1) Olivine/H₂O ratio, the rate of conversion decreases in a monotonous manner after having reached the maximum value. This seems to suggest that, under these conditions, the H₂O content of the reaction cannot keep constant the CO₂ conversion value, although at this level it is not possible to discern whether the limiting factor is Fe2+ oxidation or CO₂ reduction.

### Dependency of the Kinetics on the Energy Transfer Conditions During the Mechanical Treatment

The present study is based on the activation of the process of conversion of carbon dioxide induced mechanochemically. With such mode of activation, the energy required for triggering the chemical process is administered in the form of kinetic energy (Eₖ=½ mv²): the dependency of the reaction kinetics on the mechanical energy transferred to the reagents was evaluated by means of tests conducted under different conditions in terms of the mass of the milling bodies and the revolution speed of the motor driving the Spex mill.

As far as the former parameter is concerned, i.e. the mass of the milling bodies, several tests were carried out using three steel balls having a mass of, respectively, 1 g each, 4 g each, and 8 g each, as highlighted by the three curves shown in FIG. 6.

FIG. 6 shows, again, sigmoidal patterns for each one of the three curves referring to different experimental conditions as concerns the masses of the milling bodies employed in the tests. The time of mechanical treatment being equal, the degree of CO₂ conversion depends on the mass of the milling bodies in use, and conversion speed is also dependent on the same parameter. In general terms, it is important to point out that it is possible to determine the kinetic energy transferred during each collision, but such evaluation lies outside the present context, while still being important in view of an in-depth mechanistic survey.

Conversely, variations in the mass of the milling bodies do not seem to significantly affect the selectivity values for gaseous products like methane, ethylene and ethane, obtained during the process. In qualitative terms, these patterns follow those shown in FIG. 4a).

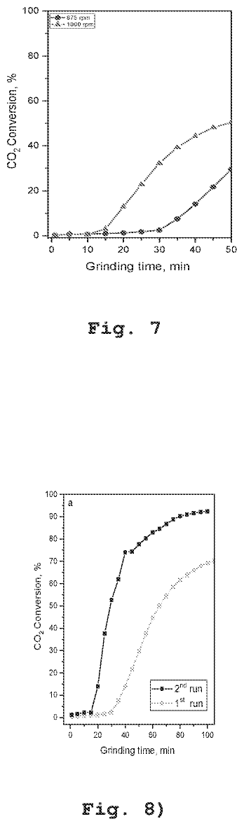

The energy transferred to the reagent species through collisions with the milling bodies can also be modulated by adjusting the revolution speed of the electric motor connected to the Spex 8000 mill. The rotation of the motor shaft of the mill imparts motion to the milling bodies and affects the frequency of the collisions between the balls and the jar walls. The tests were carried out using commercial Spex Mixer/Mill 8000 mills at revolution speeds of 875 rpm and 1,000 rpm. The results concerning the dependency of the CO₂ conversion data on the revolution speed of the motor of the mill are shown in FIG. 7.

The revolution frequency of the motor of the mill affects induction time: with a revolution speed of 1,000 rpm, the CO₂ conversion process is activated sooner than with a revolution speed of 875 rpm. In this case as well, the distribution of the final products obtained follows the pattern shown in FIG. 4a).

The study also tackled the behaviour of the systems used for the mechanically activated CO₂ conversion process when re-processed in the presence of CO₂ and H₂O. FIG. 8 shows a comparison between the experimental data curves obtained with Olivine powders at their first use (grey curve) and at their second use (black curve).

It emerges that the system is more active at the second use: induction time is shorter, and the CO₂ conversion value reaches 95%, i.e. it is higher than obtained in all tests carried out with Olivine at the “first use”.

This fact confirms the thesis that the proposed material (Olivine) is a very good candidate for use in the process of transforming CO₂ into products of high commercial value. The distribution of the products thus obtained follows the same pattern as shown in FIG. 4a).

### Morphological-Structural Characterization of the Solid Materials Before and After the Mechanochemical Reaction

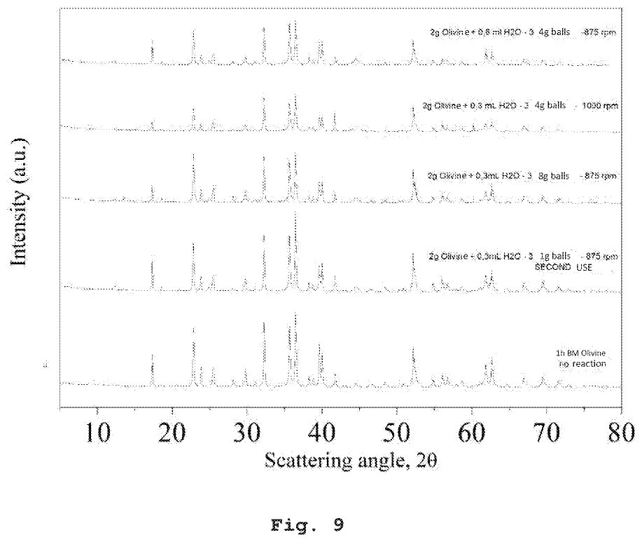

Olivine powders before (pre-grounded for 1 hour) and after the reaction were characterized by X-ray diffraction using a Rigaku SmartLab diffractometer with a rotating anode source (λ_{Cu}=1.54178 Å) and equipped with a graphite monochromator on the diffracted beam, employing a Bragg-Brentano geometry. FIG. 9 shows a sequence of X diffraction patterns concerning unprocessed Olivine and samples obtained after several mechanochemical tests under the conditions specified in FIG. 9. From the sequence of diffraction patterns for the analyzed systems summarized in FIG. 9, one can see that in the processed materials the main phases of unprocessed Olivine (Forsterite, Enstatite e Clinochlore) are preserved, with the appearance of carbonate phases such as Nesquehonite (magnesium carbonate hydrate) and Iron. Other minority phases (<1% by mass) not yet attributed, but compatible with phases such as Magnesite (magnesium carbonate), Maghemite (Fe (III) oxide) were also detected.

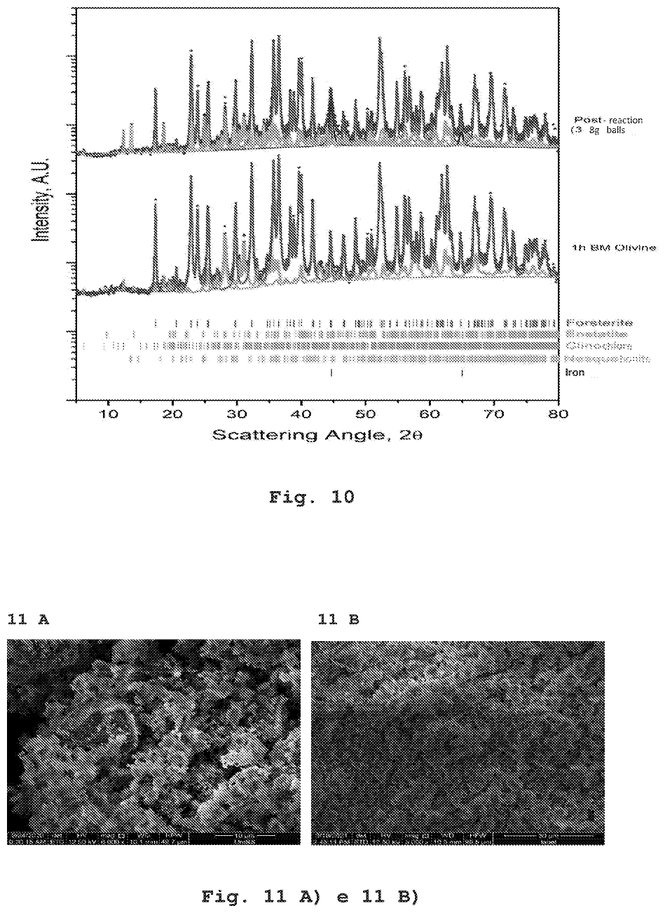

The quantitative evaluation of the abundance of the crystallographic phases in the different samples, and of the microstructural parameters, was conducted using the Rietveld refinement on method all samples under examination. FIG. 10 shows, by way of example, the X diffraction patterns concerning two samples representative of the tests carried out: Olivine after pre-treatment (lower pattern) and Olivine after mechanochemical treatment with CO₂ and H₂O (upper pattern in FIG. 10). Along with the experimental data, the figure shows the profiles concerning the structural refinement made in accordance with the Rietveld method, and the following Table 1 and Table 2 show the results of the analysis of the abundance of the crystallographic phases and of the microstructural parameters.

The X-ray diffraction pattern of unprocessed Olivine can be described through the Forsterite (90% by mass), Enstatite (8%) and Clinochlore (2%) phases, whereas the samples processed in the reactive atmosphere show the Forsterite (70-86%), Enstatite (5-7%), Clinochlore (6-15%), Nesquehonite (1-3.6%) phases, and a minority fraction of elementary Iron (1-3%) can be recognized (the latter may be due to a phenomenon of precipitation of Fe particles from the walls of the mechanochemical reactor during the process).

TABLE 1
Crystallographic data concerning the phases in the unprocessed Olivine sample, subjected to pre-grinding for 1 hour.
The quantitative evaluation was obtained by analyzing the X-ray diffraction data with the Rietveld method.

| | | AMCSD | | | | Grain | Micro |
|---|---|---|---|---|---|---|---|
| Name | Chemical Formula | code | a/Å | b/Å | c/Å | sizes nm | Strain |
| Forsterite | (Fe0.184, Mg1.816)SiO4 | 1696 | 4,763 | 10,225 | 5,994 | 103.25 | 2.33E−06 |
| Enstatite | (Fe0.155, Mg0.845)SiO3 | 1695 | 18,253 | 8,833 | 5,200 | 47.75 | 2.11E−04 |
| Clinochlore | [(Al1.84, Fe0.5, Mg4.5)Si3.16O18H8] | 4250 | 5,162 | 9,562 | 14,418 | 153.25 | 3.74E−03 |

TABLE 2
Crystallographic data concerning the phases contained in the Olivine sample after mechanochemical treatment with
CO₂ and H₂O. The quantitative evaluation was obtained by analyzing the X-ray diffraction data with the Rietveld method.

| | | AMCSD | | | | Grain | Micro |
|---|---|---|---|---|---|---|---|
| Name | Chemical Formula | code | a/Å | b/Å | c/Å | sizes nm | Strain |
| Forsterite | (Fe0.184, Mg1.816)SiO4 | 1696 | 4,762 | 10,223 | 5,993 | 106.59 | 4.40E−06 |
| Enstatite | (Fe0.155, Mg0.845)SiO3 | 1695 | 18,255 | 8,833 | 5,196 | 46.83 | 1.81E−04 |
| Clinochlore | [(Al1.84, Fe0.5, Mg4.5)Si3.16O18H8] | 4250 | 5,193 | 9,552 | 14,398 | 156.78 | 1.46E−03 |
| Nesquehonite | MgCO3•3(H2O) | 9432 | 7,767 | 5,375 | 1,213 | 119.23 | 1.93E−05 |
| α-Fe | Fe | 670 | 2,872 | 2,872 | 2,872 | 32 | 2.18E−03 |

The unprocessed materials and the processed materials (i.e. those subjected to mechanochemical reaction) were analyzed by scanning electron microscopy (SEM) using an FEI Quanta 200 microscope equipped with a Genesis XM2i Apollo 10SSD detector for EDS microanalyses.

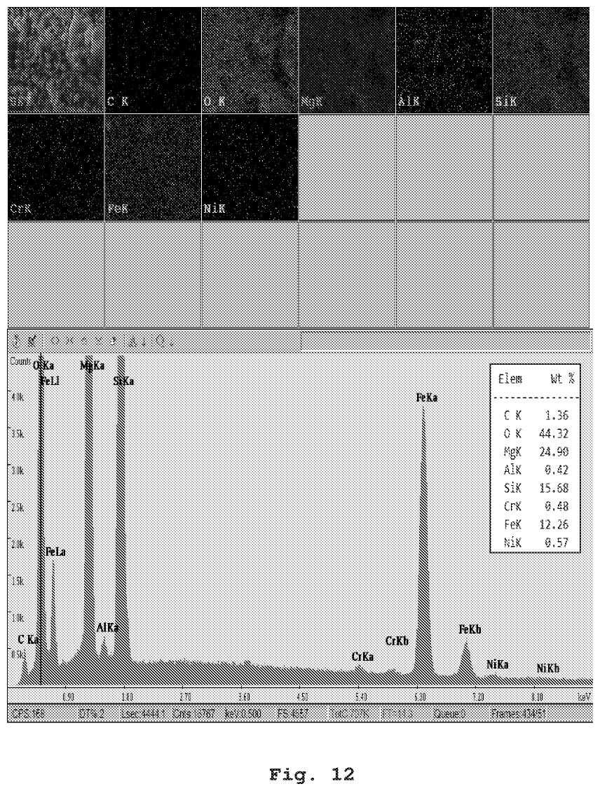

FIG. 11 shows the images obtained by means of the ETD detector, concerning the secondary electrons emitted by the samples, and illustrates the morphology of 2 samples of Olivine, respectively pre-ground for 1 hour (FIG. 11A) and processed in the presence of CO₂ and H₂O (FIG. 11B). The samples show no substantial differences, except for a smaller grain size due to prolonged exposure to grinding. In the map of the distribution of elements in the processed Olivine samples, it is possible to observe the presence of Fe, Mg, Al, Cr, Si, O, that constitute the silicate, and carbon C, the presence of which is due to the formation of carbonates following the process of mechanochemical activation in the presence of CO₂ (FIG. 12).

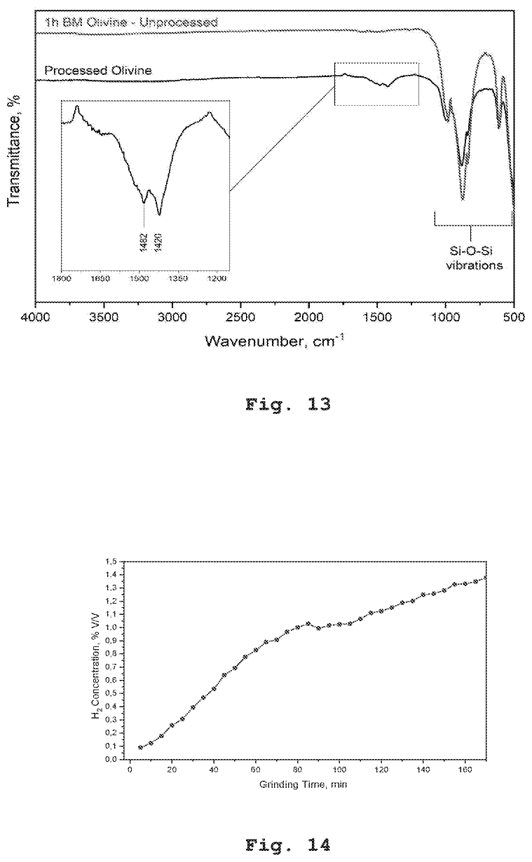

Pre-reaction and post-reaction samples were also characterized by infrared spectroscopy (FT-IR) using a Jasco FT-IR 4600 instrument in ATR mode. FIG. 13 shows the FT-IR spectra of unprocessed Olivine and Olivine subjected to the mechanochemical reaction.

The signals between 1,000 and 500 cm⁻¹ can be attributed to stretching and bending vibrations of the Si—O—Si bonds that are characteristic of silicates; such bands are easily identifiable in both spectra. In the spectrum concerning the sample of mechanically processed Olivine, it is also possible to observe the presence of two new characteristic bands centered at 1,482 and 1,420 cm⁻¹, which are compatible with the vibrations of the carbonate groups, whose appearance is due to the presence of CO₂ during the grinding process. Such signals confirm the evidence obtained from the X-ray diffraction data.

### Hydrogen Evolution

An experiment was also conducted in order to evaluate the process of evolution of hydrogen, which did not participate in the reaction with CO₂, originated from the dissociation of water induced by the mechanochemical process in the presence of Olivine. This test was carried out under flow conditions, using a mixture of 10% CO₂ balanced in Argon. The experimental setup can be summarized as follows:
2 g of Olivine pre-ground for one hour;
H₂O: 0.3 mL;
Milling bodies: 3 balls weighing 4 g each;
Revolution speed of the mill motor: 875 rpm.

The test was conducted in accordance with the same operating modes as described in the section entitled “Description of the Invention”. The experimental data concerning hydrogen evolution are shown in FIG. 14.

Hydrogen is produced starting from water by means of a dissociation process activated by the mechanochemical process in the presence of Olivine. By appropriately dosing the amount of H₂O and the Olivine/H₂O ratio in the reactor, it is possible to keep such production constant (which is a fundamental requisite) during the methanation process.

### INDUSTRIAL APPLICABILITY OF THE INVENTION

The method of the present invention has made it possible to convert carbon dioxide (CO₂) into high added value chemical compounds under continuous gas flow conditions. In particular, said method converts CO₂ into a mixture of high added value chemical compounds comprising low molecular weight hydrocarbons, mainly methane, ethylene and ethane, and products of mineral carbonation, mainly Mg and Fe carbonates. Said CO₂ conversion is achieved through a mechanochemical process and is advantageously applicable to any activity emitting into the atmosphere exhaust fumes comprising, among other possible gaseous substances, significant amounts of CO₂.

### LIST OF REFERENCES

^{[1]} https://www.climate.gov/news-features/understanding-climate/climate-change-atmospheric-carbon-dioxide
^{[2]} J. Wanga et al., International Ocean and Polar Engineering Conference, Sapporo, Japan, June 10-15 (2018)
^{[3]} https://climate.nasa.gov/effects/
^{[4]} M. Rosa et al., Journal of CO₂ Utilization, 9 (2015) 82-102
^{[5]} A.S. Agarwal et al., ChemSusChem 4 (2011) 1301-1310
^{[6]} Hydrogen in a low-carbon economy, Committee on Climate Change, London, November 2018
^{[7]} A. Álvarez et al., Chem. Phys. Chem., 18 (2017) 3135-3141
^{[8]} C. Oze et al., Geophysical Research Letters—32 (2015) L10203
^{[9]} F. Wang et al., Minerals Engineering—131 (2019) 185-19
^{[10]} M. McCollom et al, Geochem.Cosmochim. Acta 65 (2001) 3769-3778
^{[11]} V. Farina et al., Front. Energy Res., 7 (2019) 107
^{[12]} E. Turianicová et al, «A POSSIBLE WAY TO STORAGE CARBON DIOXIDE ON MECHANICALLY ACTIVATED OLIVINE (Mg, Fe)2SiO4,» INCOME 2008, Jamshedpur, India, 2008
^{[13]} F. Torre et al, Powder Technology, 364 (2020) 915-923

1. A continuous mechanochemical process under gas flow to carry out the conversion of CO₂ into a mixture of high added value chemical compounds, said mixture comprising a mixture of low molecular weight hydrocarbons, mainly methane, ethylene and ethane, and solid products of mineral carbonation, mainly Mg and Fe carbonates, said process comprising at least the following steps:
a) continuously passing a gas flow comprising, or consisting of, CO₂ through a reactor equipped with milling means, and in the presence of Olivine powder and water, in liquid or gaseous form, at from room temperature to ≤100° C.; said reactor being subjected to motion in such a way that the milling means trigger a mechanochemical reaction that, on one side, produces hydrogen from the present water, and then said hydrogen carries out the conversion of a portion of CO₂ into said low molecular weight hydrocarbons, while, on the other side, the other portion of CO₂ reacts with the transformation products of Olivine and non-reacted H₂O to give said products of mineral carbonation;
b) separating and recovering from the gas mixture exiting the reactor said low molecular weight hydrocarbons and non-reacted hydrogen obtained in step a); and
c) at the end of the reaction, separating and recovering from the reactor said solid products of mineral carbonation of Olivine obtained in step a).

2. The process according to claim 1, wherein said reactor is a mill, or a jar, equipped with milling means consisting of rotating spherical bodies (balls); said mill and said balls being made of an abrasion resistant material, hardened stainless steel.

3. The process according to claim 1, wherein the motion to which the reactor is subjected is a rotary motion at a speed of 500 rpm to 1,500 rpm.

4. The process according to claim 3, wherein said rotary motion occurs at a speed of 600 rpm to 1,400 rpm.

5. The process according to claim 1, wherein the stoichiometric ratio of Olivine to H₂O is in the range of 1:1 to 1:3.

6. The process according to claim 5, wherein the stoichiometric ratio of Olivine to H₂O is 1:2.

7. The process according to claim 1, wherein the highest measured concentrations of the low molecular weight hydrocarbons produced in step a) are 680 ppm for methane, 280 ppm for ethane, and 100 ppm for ethylene.

8. The process according to claim 7, wherein methane is the main product with a selectivity of 60%, while the selectivity values for ethane and ethylene are 20-25% and 8-10%, respectively.

9. The process according to claim 7, wherein the global yield in methane production is 50%, while it is 10-15% for ethane and about 10% for ethylene.

10. Use of the process according to claim 1 to carry out the conversion of CO₂ into a mixture of high added value chemical compounds; said mixture comprising a mixture of low molecular weight hydrocarbons, mainly methane, ethylene and ethane, and solid products of mineral carbonation, mainly Mg and Fe carbonates.
